(19) United States
(12) Patent Application Publication
(10) Pub. No.: US 2013/0252921 A1
(43) Pub. Date: Sep. 26, 2013

(54) ADHESION BARRIER CONTAINING HYALURONIC ACIDS AND L-ARGININE

(71) Applicant: HUMEDIX CO., LTD., Seoul (KR)

(72) Inventors: Ki Ho Lee, Seoul (KR); Dong Sung Lee, Uiwang (KR); Kyung Nam Kim, Hanam (KR); Jae Young Kim, Hanam (KR)

(73) Assignee: HUMEDIX CO., LTD., Seoul (KR)

(21) Appl. No.: 13/871,792

(22) Filed: Apr. 26, 2013

Related U.S. Application Data
(63) Continuation of application No. PCT/KR2010/007540, filed on Oct. 29, 2010.

(51) Int. Cl. A61K 31/198; A61K 31/728

(52) U.S. Cl. CPC A61K 31/198; A61K 31/728
USPC 514/54

(57) ABSTRACT

The present invention relates to an anti-adhesive agent containing hyaluronic acid and L-arginine, which has increased in vivo residence time and excellent anti-adhesive properties. According to the present invention, adhesion can be prevented from occurring due to surgical operation, infection and trauma.

### CROSS-REFERENCE TO RELATED APPLICATIONS

This application is continuation of International Application No. PCT/KR2010/007540 filed on Oct. 29, 2010, which is incorporated herein by reference.

### TECHNICAL FIELD

The present disclosure relates to an anti-adhesive agent containing hyaluronic acid and L-arginine, which has increased in vivo residence time and excellent anti-adhesive properties. According to the present invention, adhesion can be prevented from occurring due to surgical operation, infection and trauma.

### BACKGROUND ART

Adhesion can commonly appear in an inflammation healing process and is caused by the entanglement of cellulose which is deposited in large amounts during the formation of granulation tissue or a scar. Generally, adhesion occurs at a frequency of about 67-93% and may mostly exist even after wound healing to cause various sequelae, even though a portion thereof is spontaneously degraded. Sequelae caused by this adhesion in the case of laparoscopic surgery include intestinal dysfunction, intestinal obstruction, chronic pain and the like. Particularly, adhesion after gynecologic surgery is known to cause sterility (Eur. J. Surg. 1997, Suppl 577, 32-39).

In order to prevent this adhesion, the development of anti-adhesive agents, which cover wound sites or physically and chemically inhibit the occurrence of adhesion to the surrounding tissue by pharmacological action or the like, has been made worldwide.

Particularly, a physical anti-adhesion barrier, which is applied and attached to a wound site, has excellent effects, because it directly blocks the occurrence of adhesion. Thus, it is currently mainly used in clinical applications.

In recent years, studies on physical anti-adhesion barriers comprising bioabsorbable polymers have been actively conducted. The bioabsorbable polymers are hydrated in vivo to isolate a wound from the surrounding tissue during a wound healing period to thereby prevent the occurrence of adhesion. After completion of wound healing, the bioabsorbable polymers are naturally absorbed and removed so that they do not affect normal tissue. Various attempts to use such advantages of the bioabsorbable polymers have been made.

In examples of methods that use the bioabsorbable polymers, the use of hyaluronic acid itself as an anti-adhesive agent after surgery has been studied, and U.S. Pat. No. 4,141,973 suggests the use of hyaluronic acid. However, the use of hyaluronic acid alone shows insufficient effects due to the relatively short in vivo residence time. In addition, hyaluronic acid flows down from the surface of wounds within a short time because of its high water solubility. Due to these problems, the actual application of hyaluronic acid is problematic (Journal of Gynecologic Surgery, Vol. 7, No. 2, 97-101 (1991)).

In order to solve the above-described problem in that the anti-adhesive material hardly shows an anti-adhesive effect due to the excessively rapid degradation and removal thereof, methods for crosslinking the bioabsorbable polymer were suggested. As a specific example, EP No. 507,604 discloses a method in which a polysaccharide having a carboxyl end group is ionically bonded with polyvalent metal ions to reduce its solubility so that it can be used as an anti-adhesive agent. This method is effective in extending the time of residence in the abdominal cavity, but adhesion is not prevented even when the time of residence in the abdominal cavity is simply increased. In other words, the metal ions that are excessively used to make the metal ion salt may cause adhesion in the abdominal cavity (Eur. J. Surg. 1997, Suppl 577, 32-39). In addition, the polysaccharide forms a hard hydrogel or film by the metal ions, but the prepared film has the disadvantage of being easily broken.

Meanwhile, U.S. Pat. Nos. 5,017,229, 5,527,893, 5,760,200 and the like suggest methods for preventing adhesion using hyaluronic acid, which is a kind of polysaccharide, and carboxymethyl cellulose. When 1-ethyl-3(3-dimethylaminopropyl) carbodiimide hydrochloride (EDC) is allowed to react with hyaluronic acid and carboxymethyl cellulose, which are used as main components, a portion thereof reacts with the carboxyl end group so as to be positively charged, and the positively charged material reacts with the negatively charged carboxyl end group to spontaneously form a polyelectrolyte complex. The polyelectrolyte complex is a polymer compound having both positive and negative charges, and this compound has a hydrogel structure which is not easily degraded or dissolved because of the ionic bonds in the polymer. When the hydrogel is dried, a barrier is made, which has high absorption ability and is not easily degraded in vivo. However, because the EDC that is used in the above methods has biological toxicity, dialysis should be carried out for a long time during the preparation process. In addition, because hyaluronic acid which is used as the raw material is highly expensive, it increases the cost of the prepared film.

In addition, the films prepared by the methods described in the above-described patent documents are very brittle due their low flexibility and strength after drying and are rapidly gelled while they are hydrated. For these reasons, these films are difficult to handle and operate, because they cannot be detached and operated again after they have been applied to a biological tissue containing water (Surg. Clin. Nor. Am., 1997, 77:3, 671-688).

In addition, there have been efforts to reduce adhesion after surgery using L-arginine, pentoxifylline, methylene blue, vitamin E or the like, but these efforts have not yet exhibited sufficient effects (Kaleli B et al., Acta Obstet Gynecol Scand. 77: 377-380, 1998; Kluger Y et al., Eur J Surg.166: 568-571, 2000).

Accordingly, the present inventors have conducted studies to solve the above-described problem in that hyaluronic acid hardly shows an anti-adhesive effect due to the excessively rapid degradation and removal thereof, and as a result, have found that, when a mixture of hyaluronic acid and L-arginine is used, it has increased in vivo residence time and excellent anti-adhesive properties, thereby completing the present invention.

### SUMMARY OF THE DISCLOSURE

It is an object of the present invention to provide an anti-adhesive agent containing hyaluronic acid and L-arginine, which has increased in vivo residence time and excellent anti-adhesive properties.

### Technical Solution

The present invention provides an anti-adhesive agent containing hyaluronic acid and L-arginine.

Hyaluronic acid is a straight-chain polysaccharide in which β-D-N-acetylglucosamine and β-D-glucuronic acid are alternately bonded to each other. It has no species and organ specificity and shows excellent biocompatibility and a very high viscoelasticity even when it is transplanted or injected into a living body. Thanks to such characteristics, hyaluronic acid can be used in various applications, including an insert for treating arthritis, an anti-adhesive gel/film, a drug delivery system, a wrinkle treatment agent, and a plastic implant.

L-arginine is an essential or nonessential amino acid which is present in all organisms. When the level of L-arginine in the body is lower than the normal level, L-arginine is synthesized in the body to satisfy a physiological need, but it should necessarily be supplied to persons having a hereditary disease that reduces L-arginine.

In the case of wounds, septicemia or burns, a suitable amount of L-arginine should be taken in a dietary form to satisfy the need of L-arginine in the body. Further, it is also used as an immunonutrient for immune enhancement. In addition, it functions to neutralize ammonia which is produced in a protein synthesis process for life maintenance and in a urine formation process, and it also acts as an immune regulator together with nitric oxide, creatine, polyamines, L-glutamate, L-proline, agmatine, tetrapeptide fuftsin and the like. Moreover, it can be converted into D-glucose or glycogen when it is used as a glacogenic amino acid or degraded for biological energy production.

The use of hyaluronic acid alone as an anti-adhesive agent shows insufficient effects due to the relatively short in vivo residence time attributable to high biocompatibility and biodegradability thereof. In addition, hyaluronic acid flows down from the surface of wounds within a short time because of its high water solubility. Due to these problems, the actual application of hyaluronic acid is problematic. The inventive anti-adhesive agent containing hyaluronic acid and L-arginine overcomes the above problems and shows excellent anti-adhesive properties.

The present invention also provides an anti-adhesive agent containing hyaluronic acid and L-arginine at a weight ratio of 1:0.05-5. If the ratio of L-arginine to hyaluronic acid is less than 0.05 or more than 5, it will difficult to satisfy suitable viscosity, adhesive and application properties for the anti-adhesive agent.

Further, the present invention provides an anti-adhesive agent containing hyaluronic acid and L-arginine and having an absolute viscosity of 500-10,000 cps. If the anti-adhesive agent has an absolute viscosity of less than 500 cps, it cannot provide sufficient anti-adhesive and sticking properties due to its low viscosity, and if it has an absolute viscosity of more than 10,000 cps, it will difficult to stir and polymerize and will not be suitably distributed in an application site in tissue. For these reasons, the absolute viscosity of the anti-adhesive agent is preferably 500-10,000 cps, and more preferably 500-2,000 cps. In view of anti-adhesive, sticking and flow properties, the absolute viscosity of the anti-adhesive agent is even more preferably 1,000-2,000 cps, and most preferably 1,000-1,500 cps.

Meanwhile, examples of the formulation of the anti-adhesive agent include a solution formulation, a gel formulation and a film formulation.

When the skin or tissue is severely damaged, adhesion between tissues can occur. For this reason, recent surgical techniques have changed to techniques, which reduce the skin and tissue wound and minimize tissue damage. Due to such changes in the surgical techniques, an additional effect of preventing adhesion could also be obtained (Hepato-Gastroenterol, 1991, 38, 283). In accordance with such changes in surgical techniques, the recent tendency of development of anti-adhesive agents is toward the development of solution- or gel-type formulations (The Adhesion Prevention Opportunity, Report from MDI, 1998). The solution-type formulation refers to a method of administering a large amount of a solution formulation after performing surgery in the abdominal cavity or the pelvic cavity and is a kind of adhesion preventing method which has been attempted in various manners since adhesion in the abdominal cavity was known. The solution formulation is completely excreted in vivo within 2-3 days, but is not frequently used because of the psychological burden associated with the in vivo administration of an excessive amount of foreign matter (Eur. J. Surg. 1997, Suppl 577, 32-39). Contrary to this, the gel formulation has recently received attention, because it can effectively prevent adhesion by applying a small amount of the gel to a wound site. Until now, gel-type formulations have been developed which can be used in limited applications, including spine surgery (U.S. Pat. No. 5,605,938). This technology characterized by a composition comprising dextran sulfate as an active ingredient in addition to a protein adhesive agent was developed based on the fact that dextran sulfate is a compound that prevents the access of glial cells involved in the production of fibrous tissue. However, the above technology has a disadvantage in that it cannot be used in a surgical operation in which a surgical area is relatively large or bleeding is severe, because dextran sulfate has the property of inhibiting the coagulation of blood. Thus, the gel formulation can be used only in micro-surgical operations such as spine surgery, but has many advantages in that it is very convenient to use and can prevent adhesion to an unintended site.

In addition, in the case of the film-type anti-adhesive agent, the need to attach the anti-adhesive agent again after detaching it immediately after surgery frequently occurs. Thus, the film-type anti-adhesive agent is preferably excellent not only in initial adhesive properties, but also in re-adhesive properties.

The present invention provides a gel-type anti-adhesive agent based on hyaluronic acid having suitable viscosity and can maximize the effect of the anti-adhesive agent. However, it is to be understood that the scope of the present invention is not limited to the gel-type anti-adhesive agent and that the anti-adhesive agent may be formulated in various forms.

The inventive anti-adhesive agent containing highly biocompatible hyaluronic acid and L-arginine shows significant decreases in measurement values for the degree of adhesion and the strength of adhesion, has excellent anti-adhesive properties thanks to increased in vivo residence time, and is maintained at a suitable viscosity to exhibit very excellent flowability, application and sticking properties in tissue.

### BRIEF DESCRIPTION OF THE DRAWINGS

FIG. 1 is a graphic diagram showing the changes in absolute viscosity values before and after mixing hyaluronidase with each of Comparative Example 1 and Examples 1 to 3.

### DETAILED DESCRIPTION

Hereinafter, preferred examples will be described for a better understanding of the present invention. It is to be understood, however, that these examples are for illustrative purposes only and are not intended to limit the scope of the present invention.

### EXAMPLE 1

### Preparation of Anti-Adhesive Agent Containing Hyaluronic Acid and L-Arginine

Sodium hyaluronate and L-arginine were mixed with each other at a weight ratio of 75:25, and the mixture was mixed with distilled water at a concentration of 5.0 mg/ml and stirred at 25° C. for 1 hour, thereby preparing an anti-adhesive agent.

### EXAMPLES 2 and 3

Anti-adhesive agents were prepared in the same manner, except that the mixing ratio between hyaluronic acid and L-arginine was changed as shown in Table 1 below.

### COMPARATIVE EXAMPLES 1 and 2

Anti-adhesive agents were prepared using hyaluronic acid alone (Comparative Example 1) and L-arginine alone (Comparative Example 2). Hyaluronic acid or L-arginine was added to water at a concentration of 5.0 mg/ml, and each of the aqueous solutions was stirred at 25° C. for 1 hour.

TABLE 1

| | Hyaluronic acid (%) | L-arginine (%) |
|---|---|---|
| Comparative | 100 | — |
| Example 1 | | |
| Comparative | — | 100 |
| Example 2 | | |
| Example 1 | 75 | 25 |
| Example 2 | 50 | 50 |
| Example 3 | 25 | 75 |

### TEST EXAMPLE 1

### Oral Toxicity Test

In order to examine the oral toxicity of the inventive anti-adhesive agent, 100 ICR mice (20 g; 4-week old) were used in the test. Each of the anti-adhesive agents prepared in Comparative Examples 1 and 2 and Examples 1 to 3 was orally administered to the mice at a dose of 1 g/kg, and then mortality and abnormal syndromes such as skin rashes were examined for 2 weeks. The mortality is not included in the occurrence (%) of abnormal syndromes, and the results of the test are shown in Table 2 below.

TABLE 2

| | | Occurrence (%) of | Content of abnormal |
|---|---|---|---|
| | Mortality (%) | abnormal syndromes | syndromes |
| Untreated | 0 | 0 | — |
| group | | | |
| Comparative | 0 | 0 | — |
| Example 1 | | | |
| Comparative | 0 | 0 | — |
| Example 2 | | | |
| Example 1 | 0 | 0 | — |
| Example 2 | 0 | 0 | — |
| Example 3 | 10 | 0 | — |

As can be seen in Table 2 above, mortality and abnormal syndromes appeared in Example 3, but were not statistically significant (P>0.05). The results of Test Example 1 indicate that the anti-adhesive agents of the Examples and the Comparative Examples did not show oral toxicity, suggesting that these anti-adhesive agents have high safety.

### EXAMPLE 2

### Anti-Adhesive Effect in Abdominal Cavity

In order to examine the anti-adhesive effect of the inventive anti-adhesive agent, 4-week-old Sprague-Dawley rats (weight: about 250-300 g) were used in the test. 20 rats were used for each Comparative Example, and 10 rats were used for each Example.

The abdomen of each rat anesthetized with ether was opened, and the caecum was taken out. A wound having a size of 2×2 cm was formed in the serous membrane, and a wound having the same size was formed in the abdominal cavity membrane. Then, the abdomen was treated with each of the anti-adhesive agents of Examples 1 to 3 and Comparative Examples 1 and 2 and sutured. After 2 weeks, the rats were euthanized and the abdomen of each rat was opened. Then, the degree of adhesion and the strength of adhesion were examined. The degree of adhesion and the strength of adhesion were evaluated according to the criteria shown in Table 3 below, and the results of the evaluation are shown in Table 4 below.

TABLE 3
Degree of adhesion

| | |
|---|---|
| 0 | No occurrence of adhesion |
| 1 | One thin film-type adhesion |
| 2 | Two or more thin film-type adhesions |
| 3 | Point-type concentrated thick adhesion |
| 4 | Plate-type concentrated thick adhesion |
| 5 | Very thick adhesion having vascular |
| | vessels formed therein, or one or more |
| | plate-type adhesions |

Strength of adhesion

| | |
|---|---|
| 1 | Film-type adhesion which is detached |
| | even with very weak force |
| 2 | Adhesion which is detached with |
| | moderate force |
| 3 | Adhesion which can be detached by |
| | significant pressure |
| 4 | Adhesion which is difficult to detach |
| | or is detached by very high pressure |

TABLE 4

| | Degree of adhesion | Strength of adhesion |
|---|---|---|
| Untreated group | 4.7 | 3.4 |
| Comparative | 3.5 | 2.5 |
| Example 1 | | |
| Comparative | 4.2 | 3.1 |
| Example 2 | | |
| Example 1 | 3.0 | 2.1 |
| Example 2 | 3.4 | 2.3 |
| Example 3 | 3.8 | 3.0 |

As can be seen in Table 4 above, the anti-adhesive agents of Examples 1 to 3 have excellent anti-adhesive effects compared to those of the untreated group and Comparative Example 1 (group treated with hyaluronic acid alone). The strength of adhesion formed in the rats treated with the anti-adhesive agents also decreased, suggesting that the anti-adhesive agents are very effective in preventing the adhesion of internal organs after surgery. Particularly, in the case of Example 1 in which the ratio of hyaluronic acid to L-arginine was 75:25, the degree of adhesion was reduced to 63.8% of the untreated group, and the strength of adhesion was significantly reduced to 61.7%.

### TEST EXAMPLE 3

### Comparative Test for Hyaluronidase-Induced Degradation

Using hyaluronidase that hydrolyzes hyaluronic acid in vivo, a comparative test was performed for the degradation of the anti-adhesive agents of Comparative Example 1 and Examples 1 to 3. In the comparative test for degradation, absolute viscosity for each anti-adhesive agent was measured, and the degrees of degradation of hyaluronic acid were compared based on the difference in absolute viscosity before and after adding hyaluronidase.

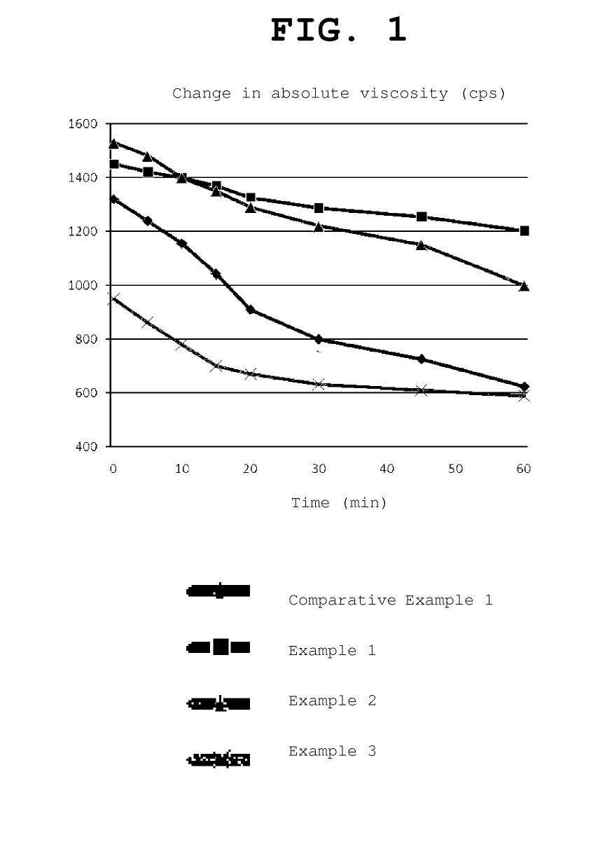

The viscosity was measured with a Brookfield Programmable Digital Viscometer DV-II+pro (Brookfield, USA) under the following conditions: spindle 3, 30 RPM, and 25° C. The results of the measurement are shown in Table 5 below and FIG. 1.

TABLE 5

| Time (min) after | Absolute viscosity (cps) | | | |
|---|---|---|---|---|
| addition of | Comparative | Example | Example | Example |
| hyaluronidase | Example 1 | 1 | 2 | 3 |
| 0 | 1320 | 1450 | 1530 | 950 |
| 5 | 1238 | 1420 | 1480 | 860 |
| 10 | 1155 | 1400 | 1400 | 780 |
| 15 | 1042 | 1370 | 1350 | 700 |
| 20 | 910 | 1325 | 1290 | 670 |
| 30 | 800 | 1286 | 1220 | 630 |
| 45 | 726 | 1253 | 1150 | 610 |
| 60 | 624 | 1201 | 1000 | 590 |

As can be seen in Table 5 above and FIG. 1, the anti-adhesive agent showed the greatest reduction in absolute viscosity. The anti-adhesive agents of the Examples showed an insignificant reduction in absolute viscosity, suggesting that these anti-adhesive agents have increased in vivo residence time to sufficient anti-adhesive effects. Particularly, the anti-adhesive agent of Example 1 showed the lowest reduction in absolute viscosity, suggesting that it has excellent flowability and anti-adhesive properties.

As described above, the inventive anti-adhesive agent containing hyaluronic acid and L-arginine has increased in vivo residence time to exhibit excellent anti-adhesive properties. In addition, it has a suitable viscosity, and thus is very excellent in flowability, application and sticking properties. Moreover, the anti-adhesive agent of the present invention can provide not only anti-adhesive effects, but also additional effects, including antibacterial, anti-inflammatory and nourishing effects, and thus can be used in various applications, including medical drugs, medical devices, cosmetics, health functional foods, foods, and the like.

1. An anti-adhesive agent containing hyaluronic acid and L-arginine.

2. The anti-adhesive agent of claim 1, wherein the weight ratio of the hyaluronic acid to the L-arginine is 1:0.05-5.

3. The anti-adhesive agent of claim 1, wherein the weight ratio of the hyaluronic acid to the L-arginine is 75:25.

4. The anti-adhesive agent of claim 1, wherein the hyaluronic acid has a viscosity of 500-10,000 cps.

5. The anti-adhesive agent of claim 1, wherein the hyaluronic acid has a viscosity of 500-2,000 cps.

6. The anti-adhesive agent of claim 1, wherein the hyaluronic acid has a viscosity of 1,000-2,000 cps.

7. The anti-adhesive agent of claim 1, wherein the hyaluronic acid has a viscosity of 1,000-1,500 cps.
